(19) United States
(12) Patent Application Publication
(10) Pub. No.: US 2012/0027822 A1
(43) Pub. Date: Feb. 2, 2012

(54) Process for Producing Rapidly Disintegrating Spheroids (Pellets), Granules and/or Mixtures Thereof

(75) Inventors: Stavros Politis, Keratea Attikis (GR); Georgios Chalkias, Velo Korinthias (GR)

(73) Assignee: SPECIFAR S.A., Ag. Varvara, Athens (GR)

(21) Appl. No.: 13/263,613

(22) PCT Filed: May 29, 2009
(86) PCT No.: PCT/EP2009/056618

§ 371 Date: Oct. 7, 2011

(30) Foreign Application Priority Data
Apr. 10, 2009 (GR) 20090100219

(51) Int. Cl. A61K 9/14; B29C 59/00

(52) U.S. Cl. 424/400; 264/15

(57) ABSTRACT

Process for producing rapidly disintegrating spheroids (pellets), granules and/or mixtures thereof, which disintegrate in less than 15 minutes. The spheroids comprise at least one active pharmaceutical ingredient, a spheronizing aid material and a solid Polyethylene glycol (PEG) with mean molecular weight (MW) of over 1,000. The process comprises a direct pelletization step in a fluidized bed rotogranulator, using aqueous binding media whilst the temperature of the process does not exceed the melting point of the solid PEG.

### FIELD OF THE INVENTION

The invention relates to the field of processes for producing rapidly disintegrating spheroids (pellets), granules and/or mixtures thereof, which include a direct pelletization step.

### BACKGROUND OF THE INVENTION

Spheroids (pellets) and granules, as solid dosage forms are considered to offer several distinct advantages over more conventional single unit systems, as they exhibit good flowability and high physical integrity which are superior qualities for filling, compression and coating applications. The therapeutic advantages of multiparticulate dosage forms such as spheroids are also well established and attributed to their in vivo performance.

One of the more recent processes for the production of spheroids is rotary processing, where the whole cycle of wet spheronization, drying, and coating can be performed in one closed system. Fluidized bed (FB) rotogranulator, centrifugal granulator, rotary fluidized bed granulator, rotary fluid bed, rotary processor and rotor granulator are some of the terms used to describe this single-pot spheronizer system, as it combines centrifugal, high intensity mixing with the efficiency of fluid bed drying. Rotary processing is an efficient, cost effective, fast, single-pot spheroid production process, during which no API loss is observed as it is incorporated in the starting materials. Few attempts have been conducted to date in order to provide manipulable immediate release formulations. Most of the proposed formulations in the bibliography are produced through the extrusion spheronization process, which demands further process steps compared to the direct pelletization processes (mixing, wetting of the powder mixtures, extrusion, spheronization of the extrudates and drying of the spheroids). Complex production processes that comprise several consecutive steps are not desirable by the pharmaceutical industry, as they are time consuming and increase potential risks for the drug product, such as cross-contamination.

Additionally, the success or failure of each step affects greatly the quality of the final spheroids and/or granules. Alternative processes such as powder and suspension layering exhibit significant API loss that typically reaches 10% due to layering alone. The cost of the API usually constitutes the most significant proportion of the cost of the finished dosage form.

In practically every process including a spheronizing step (either extrusion-spheronization or direct pelletization) Microcrystalline Cellulose (MCC) is used as spheronizing aid, in proportions larger than 20%. However, spheroids comprising MCC at these levels disintegrate slowly, a phenomenon that in most cases negatively affects the dissolution rate of the API (Kristensen et al, 2000, Direct Pelletization in a Rotary Processor Controlled by Torque measurements I. Influence of Process Variables, Pharmaceutical Development and Technology, 5 (2), 247-256, Gu et al, 2004, Wet Spheronization by Rotary Processing—A Multistage Single Pot Process for Producing Spheroids, Drug Development and Industrial Pharmacy, 30, 2, 111-123). The above problem is more intense for APIs that exhibit moderate or low aqueous solubility. Solubility, as well as permeability of APIs have been considered as the fundamental parameters of the Biopharmaceutics Classification System (BCS) which characterise and predict their in vivo performance. According to the World Health Organization (WHO), approximately 45% of drug products fall in classes II and IV of the BCS referring to low soluble drugs. The importance of aqueous solubility is also evident by the fact that all official dissolution tests require the use of aqueous media in an attempt to simulate in vivo conditions, allow in vitro-in vivo correlation and provide adequate data to assure among other in vitro tests the quality of the finished dosage form. Many official Pharmacopoeias include definitions of solubilities. For example, US pharmacopoeia (USP) categorizes the solubility of APIs as Very soluble, Freely soluble, Soluble, Sparingly soluble, Slightly soluble, Very slightly soluble, Practically insoluble or Insoluble, and the parts of solvent needed to dissolve one part of the drug substance are <1, 1-10, 10-30, 30-100, 100-1.000, 1,000-10,000, >10,000 respectively.

Therefore, for the numerous APIs that are sparingly soluble or even less soluble in water, MCC containing pellets as presented in the prior art will retard their release and thus are an inappropriate choice as immediate release formulations. Since spheroids offer clear technological and therapeutic advantages as described earlier, the development of rapidly disintegrating pellets would be beneficial as they could provide an immediate release formulation for the said APIs.

In order to overcome the above disadvantages, some formulation work has been described in the literature, mainly using super disintegrants and/or surfactants with the extrusion-spheronization process (Opitz et al, U.S. Pat. No. 6,224,909, 2001, Chukwumezie B. et al (2002), Feasibility Studies in Spheronization and Scale-up of Ibuprofen Microparticulates Using the Rotor Disk Fluid-Bed Technology, AAPS PharmaSciTech, 3, 1, 1-13., Souto C. et al, 2005, A comparative study of the utility of two superdisintegrants in microcrystalline cellulose pellets prepared by extrusion-spheronization, European Journal of Pharmaceutics and Biopharmaceutics, 61, 94-99). However the use of super disintegrants in processes that include a direct pelletization step intensifies the slow disintegration problem, while it results in a significant prolongation of the preparation phase (Kristensen et al 2002, Development of rapidly disintegrating pellets in a Rotary processor, Drug Development and Industrial Pharmacy, 28, 10, 1201-1212). On the other hand, surfactants in large quantities that are necessary to improve the dissolution rate from MCC containing pellets are known to affect the in vivo behaviour of the dosage form and especially the absorption of the API due to their interaction with the mucosal membrane.

In other efforts organic solvents such as ethanol have been used (Dreu R. et al, 2005, Physicochemical properties of granulation liquids and their influence on MCC pellets obtained by extrusion-spheronization technology, International Journal of Pharmaceutics, 291, 99-111). However the pharmaceutical industry prefers processes that do not use organic solvents for environmental and cost reasons. Other approaches employ the use of hydrophilic materials, many of which are also hygroscopic, resulting in a potential negative effect on the stability of the finished dosage form and cyclodextrins, the use of which may increase the cost of the dosage form significantly. The incorporation of hydrophillic polymers requires complicated processes (Law M. et al, 1998, Use of hydrophilic polymers with microcrystalline cellulose to improve extrusion spheronization, European Journal of Pharmaceutics and Biopharmaceutics, 45, 57-65) while other materials used are either pH dependant (pectinic acid, Tho I., Sande S. A., Kleinebudde P., 2003, Disintegrating pellets from a water-insoluble pectin derivative produced by extrusion/spheronisation, European

Journal of Pharmaceutics and Biopharmaceutics, 56, 371-380) or difficult to handle and known to induce inflammatory responses in laboratory animals (carrageenans, Thommes M. and Kleinebudde P., 2006, Use of k-carrageenan as alternative pelletisation aid to microcrystalline cellulose in extrusion/spheronisation. I. Influence of type and fraction of filler, European Journal of Pharmaceutics and Biopharmaceutics, 63, 59-67, Pharmaceutical excipients).

The use of hot melt processes has been also proposed, however a hot material typically exceeding 75° C. comes in contact with the API. It is obvious that such processes are inappropriate when APIs sensitive to thermal decomposition are used.

Typical examples of such processes are the following:

Maio M. (EP1335706 A1) describes a hot-melt process for producing microspheres, using polymers with low melting points such as PEGs. The process described in this document includes stirring of a mixture comprising the said polymer at high energy conditions and heating that leads to a temperature higher than the melting point of the meltable material. Sanghvi P. P. et al (WO0024380 A1) propose a similar hot melt approach, subjecting a mixture containing polymers such as solid PEGs to thermoforming conditions in order to produce microparticles via a direct pelletization step. Rampoldi L. and Grassano A. (WO2005055993 A2) use solid PEGs with melting point between 50 and 80° C. in mixtures that are heated until the PEG melting point in a rotogranulator in order to provide gabapentin granulates. Nagafuzi N. et al (EP0452145 A2) describe a similar approach, using thermomelting materials such as PEGs for the preparation of granules via a hot-melt centrifuge force granulation procedure.

In all four of the above patent documents, the binding medium is the melt material, while high temperatures are used.

An alternative (Vervaet C., Baert L. and Remon J. P., 1994, Enhancement of in vitro drug release by using polyethylene glycol and PEG-40 hydrogenated castor oil in pellets made by extrusion/spheronisation, international Journal of Pharmaceutics, 108, 207-212) uses low MW liquid Polyethylenglycols (PEGs). Low MW PEGs exhibit the disadvantage of increased hygroscopicity. In addition, particles containing low MW PEGs could be susceptible to partial melting and disintegration during conventional processes such as coating. Similar approaches are proposed by members of the same research team: Remon J. P. (WO9423700 A1) describes the use of liquid PEGs as solubilizers in wet granulation processes, during which mixtures of low soluble APIs and liquid solubilizers are mixed with water and are subsequently used for wet granulation and extrusion spheronization processes.

In EP1480622 A1, Remon J. P. and Vervaet K. describe an extrusion-spheronization process using solid PEGs and optionally liquid PEGs. The process they propose does not utilize aqueous media and the plasticity of the granulate that is extruded is achieved via the addition of the liquid material and/or intense kneading in the mixing zone of the extruder, at temperatures not above the melting temperature of the solid fraction of the meltable solid excipient. In the examples presented in the said document, a liquid PEG is present for this purpose. However the use of liquid PEGs is characterized as optional, although it is known according to the state of the art that the amount of binding liquid that is necessary for extrusion-spheronization techniques is higher than the amount that is necessary for wet granulation methods (Ghebre-Sellassie I. and Martin C., Pharmaceutical extrusion technology, 2003, Marcel Dekker, USA), unless the mixture is subjected to temperatures at which the meltable constituents soften or melt. In the case of the optional use of liquid PEGs the same disadvantages as the ones already mentioned could be observed in the final granule composition. In addition, this granule composition contains dextrin-containing compounds such as cyclodextrins in order to increase the dissolution rate of low soluble APIs. As already mentioned, these materials are costly.

Suzuki Y. et al (JP4187085 A) also describe an extrusion-granulation process for the production of granules containing liquid and solid PEGs with mean MW of 400-20,000. From all the above, it is clear that the introduction of a process for producing rapidly disintegrating spheroids (pellets), granules and/or mixtures thereof, which is free of the disadvantages of the approaches already mentioned was an important technical challenge, faced by the present invention.

### SUMMARY OF THE INVENTION

The present invention provides a process for producing rapidly disintegrating spheroids (pellets), granules and/or mixtures thereof, which disintegrate (when the test is performed as described in the USP) in less than 15 minutes. The terms spheroids, pellets and granules are used interchangeably, i.e. whenever one of these terms is used the other two are implied. The term spheroids used throughout the description and the claims therefore includes also pellets and granules as well as mixtures of spheroids and/or pellets and/or granules. The spheroids produced via the process of the present invention comprise one or more active pharmaceutical ingredients (APIs), a spheronizing aid material and a solid Polyethylene glycol (PEG) with mean molecular weight (MW) of over 1,000, and the process is characterized in that the direct pelletization step which is performed in a fluidized bed rotogranulator is carried out using aqueous binding media and in that the temperature of the process does not exceed the melting point of the solid PEG. According to the present invention the term “melting point” indicates the melting point of the solid PEG in each system of excipients used for the process. For example, it is known that the use of plasticizers may reduce the melting point of polymers when used in the same systems with them. In this case according to the present invention it is the modified melting point that is considered as being the melting point of the solid PEG.

The rapidly disintegrating pellets produced via the process of the present invention, preferably comprise one or more active pharmaceutical ingredients (APIs) of low to moderate aqueous solubility, which are described as sparingly soluble, slightly soluble, very slightly soluble, and practically insoluble or insoluble in the US Pharmacopoeia. One of the main advantages of the pellets is that they exhibit a dissolution rate of at least 85% after 30 minutes when tested with the methods described in the relevant official monographs of the pharmacopoeias (i.e. US Pharmacopoeia). The fast release of the API from the pellets produced via the process of the present invention is facilitated by the rapid disintegration of these pellets, which extensively increases the surface available for contact with the dissolution medium, thus facilitating drug release. Other mechanisms such as the formation of solid dispersions of the APIs in the solid PEGs or the reduction of the immobilized water accused of the slow disintegration of the typical MCC containing pellet formulations may also contribute towards the fast release of APIs with moderate to low solubilities from spheroids prepared via the process of the present invention. Besides the rapid disintegration and fast release of the API, the additional advantages of the spheroids produced via the process described in the present invention, as well as of the process itself, are multiple:
i. The spheroids produced via the process of the present invention may contain Microcrystalline Cellulose (MCC) as a spheronization aid. Thus, although the said spheroids are free of the slow disintegrating effects of this excipient, they exhibit its pharmacotechnical advantages, such as improved sphericity.
ii. The spheroids produced via the process of the present invention might provide a cushioning effect when compressed to the form of tablets, as a result of the formulation and structure of the core, which may be partially deformed, but is exceptionally resistant to fragmentation. In addition, the said spheroids exhibit the appropriate pharmacotechnical properties to withstand the common coating processes and materials, for applications related to controlled release, aesthetics, taste masking, product protection (from light and moisture) etc.
iii. The process for producing rapidly disintegrating spheroids of the present invention is exceptionally robust when additional excipients known from the state of the art are incorporated. These excipients may be selected from the groups of detackifiers, buffers, diluents, glidants, lubricants, binders etc.
iv. The process of the present invention includes a direct pelletization step utilizing aqueous media at low temperatures, lower than the melting point of the solid PEGs contained in the spheroids produced by the said process. Thus, in contrast with the hot melt approaches, it is an energy-saving process appropriate for use with thermosensitive materials. In addition, aqueous materials are safer to use and more operator-friendly than melt materials, as there is no risk of solidification of the binding medium during the pelletization process. The process of the present invention is very fast compared to conventional direct pelletization processes with aqueous media (i.e. producing spheroids comprising MCC and lactose) and thus constitutes a cost effective production process.
v. The process of the present invention allows the use of numerous potential modifications of the rotogranulation process at yields acceptable by the pharmaceutical industry. Such variations provide additional flexibility to the process of the present invention, and include the partial addition of the ingredients via a powder feeder, the dispersion or dissolution of some of the ingredients in the aqueous binding material, or the use of a wet granulation step prior to the direct pelletization step. These modifications do not affect the advantages of the spheroids manufactured via the process of the present invention and mainly intend to the increase of the yield and/or the robustness of the process. Even with these variations the process of the present invention is much less complex than the extrusion spheronization process and comprises fewer steps, while it is performed in closed systems with minimum risk of cross-contamination.
vi. The process of the present invention allows for an optional second spheronization phase during the drying step that may improve the quality characteristics of the produced spheroids. More specifically, for the production of the spheroids the approach of tangential spraying was adopted.

The process of the present invention was challenged with the use of APIs with very low solubility, as well as with low soluble APIs which are unstable when they are present in manufacturing processes with aqueous media and especially in acidic conditions. Finally, the process of the present invention was challenged with APIs that are liable to changes of their crystal habit when used in processes that utilize aqueous media. In all cases, the release profiles of these APIs from the formulations prepared according to the process of the present invention (described in the examples included in the detailed description of the invention) were compared with the relevant release profiles from spheroids prepared with MCC and lactose already known from the prior art and found to be dramatically faster. The process of the present invention did not affect the stability or the crystal habit of the tested APIs.

It is thus clear that the process described in the present invention is an attractive approach for the production of rapidly disintegrating spheroids, with simple and cost effective processes which are advantageous compared to the ones proposed in the prior art.

### BRIEF DESCRIPTION OF THE DRAWINGS

FIG. 1 presents trace plots showing the effects of the important constituents of the present invention, plus the effect of a disintegrant.

FIG. 2 depicts the XRD patterns of Esomeprazole Mg powders along with the digitized patterns of the dihydrate and trihydrate Esomeprazole forms.

FIG. 3 presents the comparative dissolution profiles of the formulations of examples 8a and 8b, which refer to pellets comprising Nimesulide, prepared as described in the prior art and according to the present invention respectively, in comparison with a commercial product incorporating surfactants.

FIG. 4 depicts the comparative dissolution profiles of the API from pellets comprising Glimepiride (Examples 8c and 8d), prepared as described in the prior art and according to the present invention respectively.

### DETAILED DESCRIPTION OF THE INVENTION

The present invention provides a process for producing rapidly disintegrating spheroids (pellets), granules and/or mixtures thereof, which disintegrate in less than 15 minutes (when the test is performed as described in the USP). The terms spheroids, pellets and granules are used interchangeably, i.e. whenever one of these terms is used the other two are implied. The term spheroids used throughout the description and the claims therefore includes also pellets and granules as well as mixtures of spheroids and/or pellets and/or granules. The spheroids produced via the process of the present invention comprise an active pharmaceutical ingredient, at least a spheronizing aid material and a solid Polyethylene glycol (PEG) with mean molecular weight (MW) of over 1000 and the process is characterized in that the direct pelletization step which is performed in a fluidized bed rotogranulator is carried out using aqueous binding media and in that the temperature of the process does not exceed the melting point of the solid PEG According to the present invention, the term “melting point” indicates the melting point of the solid PEG in each system of excipients used for the process. For example, it is known that the use of plasticizers may reduce the melting point of polymers when used in the same systems with them. In this case according to the present invention it is the modified melting point that is considered as being the melting point of the solid PEG).

The rapidly disintegrating pellets produced via the process of the present invention, preferably comprise one or more active pharmaceutical ingredients (APIs) of low to moderate aqueous solubility, which are described as sparingly soluble, slightly soluble, very slightly soluble, and practically insoluble or insoluble in the US Pharmacopoeia. The main advantage of the said pellets is that they exhibit a dissolution rate of at least 85% after 30 minutes when tested with the methods described in the relevant monographs of the official pharmacopoeias (i.e. US Pharmacopoeia). The spheroids produced via the process of the present invention comprise:
i. A drug substance, preferably in an amount that varies between 0.5-80%, more preferably between 0.5-50% and even more preferably between 0.5-30% by weight per spheroid. The process of the present invention is particularly appropriate for the formulation of APIs with low to moderate aqueous solubility, which are described as sparingly soluble, slightly soluble, very slightly soluble, and practically insoluble or insoluble in the US Pharmacopoeia.
ii. A spheronizing aid material, preferably selected from the group of

Microcrystalline Cellulose or Modified Celluloses or systems comprising one or more of these materials. The amount of the spheronizing aid material preferably varies between 5-80%, more preferably between 10-60% and most preferably between 15-55% by weight per spheroid. One of the most important features of the spheroids produced via the process of the present invention is that although they are free of the slow disintegrating effects of these excipients, they exhibit their pharmacotechnical advantages, such as improved sphericity.
iii. A solid Polyethylene Glycol with mean MW greater than 1,000, more preferably one of MW equal to or greater than 4,000 which is non-hygroscopic. The amount of the solid PEG preferably varies between 5-60%, more preferably between 10-50% and most preferably between 10-30% by weight per spheroid. Even in the case that solid non-hygroscopic PEGs are used, which are known to typically prolong the disintegration of dosage forms when incorporated at ratios over 5%, the function of these compounds in the spheroids produced via the process of the present invention was surprisingly found to enhance the disintegration properties and the release profile of the API from these formulations.

The fast release of the API from the pellets produced via the process of the present invention is facilitated by the rapid disintegration of these pellets, which extensively increases the surface available for contact with the dissolution medium, thus facilitating drug release. Other mechanisms that may also contribute towards the fast release of APIs with moderate to low solubilities from spheroids prepared via the process of the present invention are the formation of solid dispersions of the APIs in the solid PEGs or the reduction of the immobilized water accused of the slow disintegration of the conventional MCC containing pellet formulations. The solid PEGs also regulate the integrity of the spheroids during compression, to an extent known from the state of the art (Vergote et al, 2002, Wax beads as cushioning agents during the compression of coated diltiazem spheroids, European Journal of Pharmaceutical Sciences, 17, 145-151, Nicklasson et al, 1999a, Modulation of the tabletting behaviour of microcrystalline cellulose spheroids by the incorporation of Polyethylene glycol, European Journal of Pharmaceutical Sciences, 9, 57-65). As a result, the spheroids produced via the process of the present invention might provide a cushioning effect when compressed to the form of tablets, as a result of the formulation and structure of the core, which may be partially deformed, but is exceptionally resistant to fragmentation. In addition, the said spheroids exhibit the appropriate pharmacotechnical properties to withstand common coating processes and materials, for applications related to controlled release, aesthetics, taste masking, product protection (from light and moisture) etc., such as coatings comprising HPC, HPMC, PVA, PVAP, Methacrylate copolymers, EC etc. Finally, the spheroids produced via the process of the present invention are robust during processes known from the state of the art, such as filling in hard gelatine capsules and compression into tablets.

The spheroids produced via the process of the present invention may optionally further contain:
i. A detackifier or anticaking agent, such as talc or aerosil, preferably a detackifier that is non-hygroscopic. The amount of the detackifier is preferably less than 30% and more preferably less than 10% by weight per spheroid. The use of the detackifier does not significantly affect the disintegration of the spheroids and the release of the API from the formulation. The inclusion of such a detackifier into the formulation however may further increase the yield of the process, by reducing sticking on the surface of the product chamber.
ii. Other common excipients known from the state of the art, such as fillers or diluents, lubricants and glidants, binders and disintegrants,. With regard to the fillers, lactose is the diluent of choice; however other carbohydrates such as mannitol or sorbitol, inorganic materials, such as bicalcium phosphate, etc. may be used. The use of binders is not necessary for the formation of spheroids using the process of the present invention; however their use may further accelerate the process. Disintegrants may be also added, however they do not affect the disintegration properties of the spheroids produced via the process of the present invention. Their presence may prolong the duration of the direct pelletization step of the said process which in some cases is in favour of the quality characteristics of the produced spheroids, as longer spheronization times are achieved. This phenomenon was studied with the use of sodium alginate at different levels.

As already mentioned, the process of the present invention includes a direct pelletization step utilizing aqueous media at low temperatures, lower than the melting point of the solid PEGs contained in the spheroids produced by the said process. Thus, in contrast with the hot melt approaches, it is an energy-saving process appropriate for use with thermosensitive materials. In addition, aqueous materials are safer to use and more operator-friendly than melt materials, as there is no risk of solidification of the binding medium during the pelletization process.

The process of the present invention is very fast compared to conventional direct pelletization processes with aqueous media (i.e. producing spheroids comprising MCC and lactose) and thus constitutes a cost effective production process. In addition, the process of the present invention allows for an optional second spheronization phase during the drying step that may improve the quality characteristics of the produced spheroids.

A thorough description of the direct pelletization process is made in “Pharmaceutical Pelletization Technology” (Gheblre-Sellassie, 1989). Typically, the approach of direct pelletization via tangential spraying is adopted. A fluid bed device equipped with a rotor is used, consisting of a cylindrical chamber with a solid disk at its base. A gap exists at the perimeter of the disk, through which the process air is drawn. The rotation speed of the disk may vary. The spraying nozzle is positioned on the side of the product container and beneath the surface of the material, so that the binding material is sprayed tangentially to the tumbling particles. More recent approaches propose the use of the top spray technique or a combination of top spray and tangential techniques.

As a result, through the process, three forces combine to provide a pattern that is best described as a spiralling helix. These three forces are the centrifugal force, the force of the fluidized air through the gap and gravity and cause the product to move towards the wall of the chamber, upwards and inwards (back to the rotor), respectively. This spiral “rope-like” motion is considered responsible for the high efficiency of the centrifugal equipment. It is of great importance that the flow pattern is smooth and no mass transfer of the product without rotation should be observed.

The process of the present invention allows the use of numerous potential modifications of the rotogranulation process at yields acceptable by the pharmaceutical industry. Such variations provide additional flexibility to the process of the present invention, and include for example the partial addition of the ingredients via a powder feeder, the dispersion or dissolution of some of the ingredients in the aqueous binding material, or the use of a wet granulation step prior to the direct pelletization step. These modifications do not affect the advantages of the spheroids manufactured via the process of the present invention and mainly intend to the increase of the yield and/or the robustness of the process. Even with these variations the process of the present invention is much less complex than the extrusion spheronization process and comprises fewer steps, while it is performed in closed systems with minimum risk of cross-contamination.

In a preferred embodiment of the present invention the process comprises the following steps and parameters:
The rotor speed ranges between 400-1,400 rpm when the rotor's diameter is approximately 30 cm and the loading ranges between 500-1,500 g. The speed may be adjusted for given disks of different diameters and loads via the constant Froude number or the constant disk peripheral velocity, in a way known to the man skilled in the art. The rotor speed may be altered during the process, enabling a slow-fast-slow, or a fast-slow procedure.
Typical fluidized air flows during the direct pelletization step range between 50 and 80 m³/h. The fluidized air temperature is typically set at 30-40° C., resulting in product temperatures of 16-20° C. during spraying of the binding material.
The addition of the binding material is performed at a rate ranging between 0.02-0.05 g/min/g_{mixture in rotor}, in order to maintain appropriate humidity contents in the product chamber of the rotogranulator. The addition rate of the binding material may be altered during the process. The binding material may be deionized water. The binding material may also be pH adjusted via a manner known from the state of the art, such as the use of pharmaceutically acceptable acidic or alkaline constituents and/or buffer solutions.
The constituents may be submitted to further treatment such as milling, sieving, blending, wet and dry granulation, drying and other pharmaceutically acceptable process steps known to the man skilled in the art, aiming at reducing filter blocking during the initial stages of rotogranulation, enhancing the uniformity and/or the particle size of the powder mixture used for rotogranulation and/or the yield of the direct pelletization step. In detail, wetting the powder mixture reduces the amount of fluidized material that may block the filters during the initial stages of the direct pelletization step. This is important when materials of small particle size are used and may lead to an increase of the yield of the process. The wetting step should not result in large particles that will act as large nuclei during the direct pelletization step, as this affects the size and quality of the pellets obtained via the said step. Wetting of the materials is performed via a wet granulation process, preferably using a spraying device and a conventional granulation or a high shear apparatus.
The ingredients of the formulation may be partially supplied via the powder feeder apparatus. The powder feeding rate ranges between 0.01-0.04 g/min/g_{mixture in rotor}.
Drying is optionally performed in the fluidized bed rotogranulator. The product temperature is increased and/or the disk rotation speed is modified and/or the disk opening is increased and/or the fluidized bed volume is increased during the drying phase, in order to achieve drying of the spheroids. The extent of drying in the rotogranulator may be appropriately adjusted according to the ingredients of formulation and the process parameters. Further drying may be performed in trays, in ventilated ovens and/or in fluid bed apparatus.
Using the process of the present invention a second spheronization phase is feasible during drying, at product temperatures close to 38-40° C. that are significantly lower than the ones met in hot-melt applications. Using the process described herein, drying cycles of more than 40 minutes are feasible, during which the spheroids may be further spheronized due to the second spheronization phase. Even in the case of fluid bed drying the pellets produced with the process of the present invention are robust to attrition that is observed during drying of the conventional MCC:Lactose formulations. This is important, as according to the prior art, if the acceptable residual moisture (or solvent content) is not achieved in less than 10 minutes after the completion of the spraying step, a tray drying process step should be considered in order to minimize the erosion of the spheroid surface.

As already mentioned, modifications of the direct pelletization technique as described herein include the addition of the component materials from a powder feeder or dispersed in the binder liquid, however these are simply modifications of the direct pelletization technique and inert cores are not used in any step of the production step. Even in the case that an API is added through the powder feeder or suspended in the binding material, the addition is performed co-instantaneously with the formation of the spheroids, in a way that the API is present inside the core of the spheroids, not only layered on the cores. The process by which inert cores are prepared through a direct pelletization process, dried to the extent that further spheroid formation is not feasible and then coated with drug containing layers is not included in the scope of the present invention.

The process of the present invention is not thermoforming and does not utilize high temperatures, or liquid PEGs, or organic solvents.

One preferred embodiment according to the present invention is a process for producing rapidly disintegrating spheroids comprising:
i. An API, in an amount varying between 0.5-30% by weight per spheroid.
ii. A spheronizing-aid excipient such as microcrystalline cellulose in an amount varying between 15-55% by weight per spheroid.
iii. A solid PEG such as PEG 4000, in an amount varying between 10-30% by weight per spheroid.
iv. A detackifier, such as talc, in an amount varying between 2.5-5% by weight per spheroid.

The process is characterized in that the direct pelletization step which is performed in a fluidized bed rotogranulator is carried out by using aqueous binding media and in that the temperature of the process does not exceed the melting point of the solid PEG.

The following examples illustrate the present invention without limiting its scope.

### EXAMPLES

The process of the present invention was challenged with:
i. The use of APIs of low aqueous solubility (Examples 1, 2, 3, 4, 7, 8), at different dosages. Especially in example 8 two practically insoluble APIs were used, such as Nimesulide and Glimepiride.
ii. The use of low soluble APIs which are unstable when they are present in manufacturing processes with aqueous media and especially in acidic conditions. For example, the present invention may be applied to APIs that are characteristic representatives of the group of Proton Pump Inhibitors (PPIs). This group comprises substituted benzimidazoles and its typical representatives include Omeprazole (very slightly soluble), Esomeprazole (slightly soluble or insoluble, depending on the salt that is used), Lansoprazole (practically insoluble), Rabeprazole (very soluble), Pantoprazole (freely soluble) and their pharmaceutically acceptable salts. Two representatives of low solubility were selected (Omeprazole, Esomeprazole, Examples 1, 2, 3, 4, 7). These drugs are typically formulated as immediate release dosage forms coated by various coating layers at least one of which is an enteric coating. The cores of the commercially available spheroids are mainly prepared via complicated and time consuming processes such as powder layering of inert seeds or extrusion spheronisation. Therefore, providing a simple spheroid manufacturing process such as the one disclosed herein results in major advantages for the industrial production of rapidly disintegrating spheroids.
iii. Finally, the use of an API salt that is liable to changes of its crystal habit when used in processes that utilize aqueous media (Example 7).

In all cases, the release profiles of these APIs from the formulations prepared according to the process of the present invention were compared with the relevant release profile from MCC:lactose spheroids already known from the prior art and found to be significantly faster. The process of the present invention did not affect the stability or the crystal habit of the tested APIs.

Examples 5 and 6 describe the application of the process of the present invention with placebo trials. Example 5 explored the necessity of the binding material in order to provide spheroids, while example 6 describes a design space within which the two main constituents of the spheroids produced via the process of the present invention (spheronizing aid excipient and solid PEG) vary together with a disintegrant (Sodium alginate), testing the effect of each constituent on responses such as the disintegration of the spheroids, spheroid size and sphericity.

In all the following examples, the monohydrate form of lactose is used.

### Example 1

Example 1 illustrates the slow release profile of a spheroid formulation consisting of a diluent (lactose) a spheronizing aid material (MCC) and an API (Omeprazole-Model drug A), as well as the effect of the incorporation of a super disintegrant (Crospovidone or Crosscarmellose sodium). This example is presented in order to compare the spheroids of the prior art with the spheroids produced via the process of the present invention.

Example 1a

111.5 g Omeprazole salt, 444.25 g lactose, and 444.25 g microcrystalline cellulose, were accurately weighed. In order to optimize the properties of the powder mixture in terms of uniformity prior to the pelletization process, a mixing step was adopted. The API, the spheronizing aid-excipient (MCC), and the diluent (Lactose) were sieved through an appropriate sieve (900 μm) and were blended for 5 min, until a uniform mixture was prepared. The rotogranulator was preheated at appropriate temperature, resulting in initial product temperatures of about 30° C., rapidly reducing to 18-24° C. during the process. The powder mixture was added and the rotogranulation process began by spraying the binding material, typically water, while the rotor speed was set at an appropriate value, ranging between 200-1,400 rpm at different process steps. The binding material addition rate ranged between 0.03-0.05 g/min/g_{mixture in rotor}, or alternatively between 30-50 g/min for every 1,000 g of powder mixture in the rotor. The binding material was deionized water with a pH value of 10 adjusted with NaOH solution. The powder mixture was rotogranulated using approximately 1,800 ml of distilled water. The formed spheroids were dried until the relative humidity in the rotogranulator was less than 15%. The inlet air temperature was set at a value allowing the drying of the spheroids (50-60° C., resulting in a product temperature of approximately 35-40° C.).

### Example 1b

223.0 g Omeprazole salt, 388.50 g lactose, and 388.50 g microcrystalline cellulose, were formulated into spheroids using a similar process as in Example 1a. Approximately 1,300 ml of distilled water were used.

### Example 1c

111.5 g Omeprazole salt, 419.25 g lactose, 419.25 g microcrystalline cellulose and 50.0 g crosspovidone, were formulated into spheroids using a similar process as in Example 1a. More than 3,700 ml of distilled water were used, while the size of the spheroids did not increase, despite the successive increase in the supply rate of the binding liquid.

### Example 1d

111.5 g Omeprazole salt, 419.25 g lactose, 419.25 g microcrystalline cellulose and 50.0 g, Crosscarmellose Sodium, were formulated into spheroids using a similar process as in Example 1a. More than 3,000 ml of distilled water were used, while the size of the spheroids did not increase, despite the successive increase of the supply rate of the binding liquid.

All the spheroids produced according to the formulations described in example 1 did not disintegrate at 37° C. The release of the API (test according to the dissolution analysis described in the current Pharmacopoeias, enabling USP apparatus II) was very slow and via diffusion through the core of the spheroids. Less than 40% was released in 30 min, while a slight increase (approximately 10%) was noticed when the API content was doubled (Example 1b), although the solubility of this model drug is low (very slightly soluble in water). This denotes that the core can significantly prolong drug release, an outcome that is in compliance with the prior art. In addition, during drying attrition occurred at relative humidity (RH) conditions less that 20%, which was identified by the presence of fine powder in the rotor compartment. Spheronizing of the spheroids was feasible only during the binding material spraying phase.

### Example 2

Example 2 illustrates the formulation of the present invention and especially the effect of the addition of the solid PEG.

### Example 2a

111.5 g Omeprazole salt, 444.3 g lactose, 222.1 g microcrystalline cellulose and 222.1 g Polyethylene Glycol (PEG) 4000 were formulated into spheroids using a similar process as in Example 1a. Less than 700 ml of distilled water were used, while the size of the spheroids increased as more binding material was sprayed.

The process was much faster resulting in uniform spheroids. The yield of the process exceeded 85% in the useful range.

### Example 2b

111.5 g Omeprazole salt, 444.3 g lactose, 322.1 g microcrystalline cellulose and 122.1 g Polyethylene Glycol (PEG) 4000 were formulated into spheroids using a similar process as in Example 1a. Less than 600 ml of distilled water were used.

### Example 2c

111.5 g Omeprazole salt, 222.1 g lactose, 444.3 g microcrystalline cellulose and 222.1 g Polyethylene Glycol (PEG) 4000 were formulated into spheroids using a similar process as in Example 1a. Less than 600 ml of distilled water were used.

### Example 2d

222.3 g Omeprazole salt, 333.5 g lactose, 222.1 g microcrystalline cellulose and 222.1 g Polyethylene Glycol (PEG) 4000 were formulated into spheroids using a similar process as in Example 1a. Less than 500 ml of distilled water were used.

The formulations of examples 2b and 2c exhibited a 30-40% increase in the release rate compared to the formulations described in example 1. This behaviour was not expected, as the high MW PEGs tend to prolong the disintegration of solid oral dosage forms. Formulations of examples 2a and 2d rapidly released the API. The drug substance was quantitatively released (>85%) during the first 10 minutes of the dissolution test. Surprisingly, the amount of binding material was also reduced, resulting in faster processes of spheroids' production. This attribute is desired for the production of spheroids comprising unstable APIs such as the representatives of the group of PPIs used herein. In addition, during drying and at RH values of less than 15%, a second spheronization phase occurred, resulting in spheroids of improved quality characteristics in terms of sphericity and surface roughness. The product temperature at which this behaviour was observed did not exceed 38-40° C.

### Example 3

Example 3 illustrates the formulation of the present invention. Both the effect of the solid PEG and the detackifier are presented.

### Example 3a

111.5 g Omeprazole salt, 394.3 g lactose, 222.1 g microcrystalline cellulose, 222.1 g Polyethylene Glycol (PEG) 4000 and 50.0 g Talc were formulated into spheroids using a similar process as in Example 1a. Surprisingly, less than 550 ml of distilled water were used, although a practically insoluble material like talc was incorporated.

### Example 3b

110.0 g Omeprazole salt, 165.0 g lactose, 375.0 g microcrystalline cellulose, 300.0 g Polyethylene Glycol (PEG) 4000 and 50.0 g Talc were formulated into spheroids using a similar process as in Example 1a. Surprisingly, less than 500 ml of distilled water were used, although the content of the spheronizing aid material (MCC) was increased and a practically insoluble material like talc was incorporated.

### Example 3c

Similar to Example 3b, adding 750 ml of distilled water as a binding material. The system reached equilibrium where the increase of the spheroid size was slow, further facilitating the production of spheroids of the desired size.

### Example 3d

110.0 g Omeprazole salt, 90.0 g lactose, 450.0 g microcrystalline cellulose, 300.0 g Polyethylene Glycol (PEG) 4000 and 50.0 g Talc were formulated into spheroids using a similar process as in Example 1a. Surprisingly, less than 600 ml of distilled water were used.

All formulations in example 3, released the API quantitatively within 30 min, while the disintegration of the spheroids was evident through macroscopic observation. The process of the present invention is able to provide rapidly disintegrating spheroids, without the use of superdisintegrants and/or surfactants and/or liquid PEGs or hot-melt approaches, at low temperatures below the melting point of the solid PEGs used. The process of the present invention is robust when different levels of the spheronizing aid excipient are used. This excipient is liable for the slow disintegration of spheroids as described in the prior art. Via the proposed process, the spheronizing aid excipient may be used at quantities that allow the production of spherical spheroids, without deteriorating the disintegration of the spheroids and the dissolution rate of the API from the resulting solid oral dosage forms.

Spheroids of example 3a were coated in a rotary processor using an HPMC-based film coating (Opadry Clear®), with solids content of 8%, at a spray rate of approximately 0.02 g/min/g_{spheroids in the product chamber} at a temperature of 37-40° C. A second methacrylates-based enteric coating (opadry acryleze®) was applied using a similar process.

The compositions of examples 2 and 3 were found to be stable at accelerated conditions (temperature of 40° C. and relative humidity of 75% RH). In detail, uncoated spheroids were studied and no significant decrease concerning the API quantitative determination was noticed.

### Example 4

Example 4 explores the robustness of the formulation of the present invention to alternative modifications of the direct pelletization process. In some cases placebo trials are presented.

### Example 4a

As example 3b, where PEG 4000 and talc are provided from the powder feeder, at a rate of approx. 0.01-0.04 g/min/g_{mixture in rotor}.

### Example 4b

As example 3b, where MCC and talc are provided from the powder feeder, at a rate of approx. 0.01-0.04 g/min/g_{mixture in rotor}.

### Example 4c

As example 3b, where Lactose and talc are provided from the powder feeder, at a rate of approx. 0.01-0.04 g/min/g_{mixture in rotor}.

### Example 4d

As example 3b, where all the constituents are mixed and 30-50% w/w of the mixture are provided from the powder feeder, at a rate of approx. 0.01-0.04 g/min/g_{mixture in rotor}.

### Example 4e

As example 3b, where PEG 4000 is dispersed in the binding material in a proportion ranging between 20-60% w/w.

### Example 4f

As example 3b, where PEG 4000 is milled.

### Example 4g

As example 3b, where the binding material is heated at 60-75° C.

### Example 4h

As example 3b, enabling Mg stearate 1% as lubricant and 2% Aerosil as glidant.

### Example 4i

111.5 g Omeprazole salt, 400.0 g lactose, 250.0 g microcrystalline cellulose, 250.0 g Polyethylene Glycol (PEG) 4000, 50.0 g talc, 50.0 g Sodium Alginate were formulated into spheroids using a rotogranulation process at a spray rate of approximately 27.5 g/min, at 30° C. The rotation speed was 1,300 rpm, while drying was performed at 55-60° C. An amount of MCC equal to 1% of the whole powder mixture was added via the powder feeder at the end of the spraying step. The particle size of PEG was smaller than 300 μm.

All the above variations of the process provide spheroids of comparable quality characteristics, thus proving that the formulation and the manufacturing process are robust to such modifications. The feasibility of performing a second spheronization phase was observed with during all trials. In all cases, the release rate of the API from the formulations was fast (>85% in 30 minutes for Omeprazole salt).

### Example 5

Example 5 explores the necessity of the binding material in order to provide spheroids.

### Example 5a

A placebo trial resembling example 2a, using an aqueous binding material containing 3% HPC of low viscosity.

### Example 5b

A placebo trial incorporating 500.0 g lactose, 300.0 g microcrystalline cellulose and 200.0 g Polyethylene Glycol (PEG) 4000, using an aqueous binding material containing 3% HPC of low viscosity.

The formulations described in example 5 provided spheroids faster and using smaller amounts of binding material (less than 400 ml of sprayed material) compared to the case that the binder was not used, however these spheroids were brittle and less resistant to crushing compared to spheroids that did not contain the binder (HPC). Thus the use of a binder does not offer any advantages to the formulation concerning the basic reasons for which a binder may be used.

### Example 6

Example 6 explores a design space within which the two main constituents of the spheroids produced via the process of the present invention (spheronizing aid excipient and solid PEG) may vary and the resulting responses of disintegration, spheroid size and sphericity. Moreover the effect of the addition of a disintegrant (Sodium Alginate) is studied.

In order to explore this design space a D-optimal mixture design was selected, the different constituents varying as follows (Table 1):

TABLE 1
Excipients of the formulations produced via the process of the present
invention and quantities thereof.

| | Spheronizing | | Disintegrant | | |
|---|---|---|---|---|---|
| | aid | Solid PEG | C: Sodium | Detackifier | Diluent |
| No | A: MCC | B: PEG 4000 | Alginate | Talc | Lactose |
| 1 | 0.3500 | 0.2000 | 0.0250 | 0.0500 | 0.3750 |
| 2 | 0.2500 | 0.2750 | 0.0500 | 0.0500 | 0.3750 |
| 3 | 0.3000 | 0.2000 | 0.0750 | 0.0500 | 0.3750 |
| 4 | 0.2750 | 0.2750 | 0.0250 | 0.0500 | 0.3750 |
| 5 | 0.2500 | 0.2500 | 0.0750 | 0.0500 | 0.3750 |
| 6 | 0.2988 | 0.2388 | 0.0375 | 0.0500 | 0.3750 |
| 7 | 0.3250 | 0.2000 | 0.0500 | 0.0500 | 0.3750 |
| 8 | 0.3250 | 0.2000 | 0.0500 | 0.0500 | 0.3750 |
| 9 | 0.2738 | 0.2575 | 0.0438 | 0.0500 | 0.3750 |
| 10 | 0.3175 | 0.2200 | 0.0375 | 0.0500 | 0.3750 |
| 11 | 0.3000 | 0.2000 | 0.0750 | 0.0500 | 0.3750 |
| 12 | 0.3500 | 0.2000 | 0.0250 | 0.0500 | 0.3750 |
| 13 | 0.2750 | 0.2750 | 0.0250 | 0.0500 | 0.3750 |
| 14 | 0.2500 | 0.2750 | 0.0500 | 0.0500 | 0.3750 |

All the formulations of table 1 disintegrated rapidly in less than 2.5 minutes.

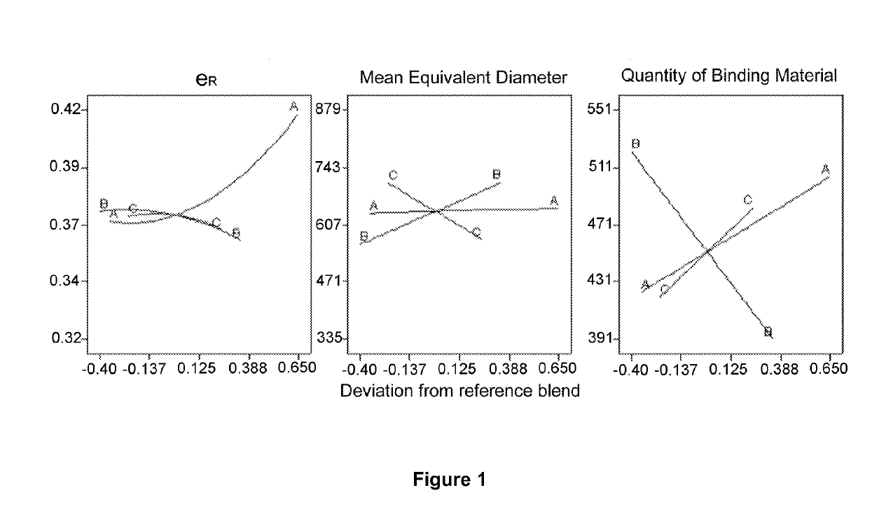

The plots presented in FIG. 1 are called trace plots and represent the effect of each constituent on a specific quality characteristic, in this case sphericity, mean equivalent diameter and quantity of binding material necessary for the formation of spheroids of equal size. The lines represent the effect of changing each mixture component while keeping all others to a constant ratio. The response is plotted while moving along an imaginary line from a reference blend to the vertex of the component being incremented. The default reference blend is the centroid of the design. As the amount of this component increases, the amount of all the other components decreases, but their ratio to one another remains constant. Larger slopes of the lines in the trace plot indicate a more significant effect of the relevant constituent to the response.

The following conclusions may be drawn from the plots of FIG. 1:

The sphericity (as denoted by the index e_{R}) increases as the MCC content of the formulation is increased. Thus the role of MCC as a spheronizing aid material is retained in the formulation of the present invention. The solid PEG and the disintegrant do not affect the sphericity significantly.

The size of the spheroids (as denoted by the mean equivalent diameter) increases as the solid PEG content increases and decreases as the proportion of the disintegrant increases. The quantity of binding material necessary for the preparation of spheroids increases as the proportions of the spheronizing aid excipient (MCC) and the disintegrant increase. On the contrary, an increase in the proportion of the solid PEG reduces the quantity of the binding material, resulting in shorter manufacturing processes.

### Example 7

Example 7 refers to the outcomes of testing the process of the present invention with an API (Esomeprazole Magnesium Dihydrate-Model Drug B), which exhibits the following deficiency: It is a low soluble API, susceptible to polymorphic changes due to the process.

### Example 7a

110.0 g Esomeprazole Magnesium Dihydrate, 340.0 g lactose, 250.0 g microcrystalline cellulose, 250.0 g Polyethylene Glycol (PEG) 4000 and 50.0 g Talc were formulated into spheroids using a rotogranulation process at a spray rate of approximately 27.5 g/min, at 30° C. Drying was performed at 55° C., resulting in product temperatures of approximately 40-45° C.

### Example 7b

Esomeprazole Magnesiun Dihydrate used in example 7a was micronized. In this form the said material is adhesive and thus difficult to formulate, while it tends to block the filters of the rotogranulator during the initial steps of the rotogranulation process. In order to overcome these difficulties, a powder mixture consisting of 200.0 g Esomeprazole Magnesium Dihydrate, 150.0 g lactose, 350.0 g microcrystalline cellulose and 200.0 g Polyethylene Glycol (PEG) 4000 was used. These materials were mixed and wet granulated in a high shear mixer-granulator, up to a humidity content of 15%. The wet granulation step was optimized with the use of a binary spraying nozzle, in order to provide fine wet material and not granules or large agglomerates. The wet mixture was mixed with 40.0 g talc, 15.0 g colloidal silicon dioxide and 25.0 g Sodium Alginate and inserted in the fluidized bed rotogranulator and was formulated into spheroids using a rotogranulation process at a spray rate of approximately 25 g/min, at 30° C. Following this variation of the process of the present invention the filters of the apparatus were completely free from any material. Additional 20.0 g of Talc were added at the end of the direct pelletization step and the spheronization was continued for 5 more minutes in the product chamber of the fluidized bed rotogranulator. Further drying was performed in a fluid bed apparatus, due to the adhesive properties of the API at a specific level of humidity content. Drying was performed at 55° C.

### Example 7c

The efficacy of the wet granulation step described in example 7b was challenged by increasing the content of the fine API, using a formulation comprising 250.0 g Esomeprazole Magnesium Dihydrate, 100.0 g lactose, 350.0 g microcrystalline cellulose, 200.0 g Polyethylene Glycol (PEG) 4000, 40.0 g talc, 15.0 g colloidal silicon dioxide and 25.0 g Sodium Alginate. The wet granulation process (using the API, lactose, microcrystalline cellulose and PEG 4000) and the direct pelletization step (adding the rest of the materials) were performed in a similar way as described in example 7b. No blocking of the filters was observed during the direct pelletization step.

### Example 7d

In order to improve the sphericity of the Esomeprazole containing pellets of examples 7a-7c, the MCC content was increased: 200.0 g Esomeprazole

Magnesium Dihydrate, 500.0 g microcrystalline cellulose and 200.0 g Polyethylene Glycol (PEG) 4000 were wet granulated in a similar way as described in example 7b. The wet mass was mixed with 40.0 g talc, 15.0 g colloidal silicon dioxide and 25.0 g Sodium Alginate and the mixture rotogranulated using a direct pelletization step. Lactose was not included in this formulation.

### Example 7e

The formulation of example 7d was modified by increasing the API content at 25%. 250.0 g Esomeprazole Magnesium Dihydrate, 450.0 g microcrystalline cellulose and 200.0 g Polyethylene Glycol (PEG) 4000 were wet granulated in a similar way as described in example 7b. The wet mass was mixed with 40.0 g talc, 15.0 g colloidal silicon dioxide and 25.0 g Sodium Alginate and the mixture was rotogranulated using a direct pelletization step. Lactose was not included in this formulation.

### Example 7f

The process of example 7e was modified by splitting the amount of the spheronizing aid material in two portions, one of which was excluded from the wet granulation step and added as an external phase before the direct pelletization step. The purpose of this change was to further prolong the direct pelletization step and resulted in further increasing the yield of the process. 250.0 g Esomeprazole Magnesium Dihydrate, 200.0 g microcrystalline cellulose and 200.0 g Polyethylene Glycol (PEG) 4000 were wet granulated in a similar way as described in example 7b. The wet mass was mixed with the MCC portion excluded from the wet granulation step and 40.0 g talc, 15.0 g colloidal silicon dioxide and 25.0 g Sodium Alginate and the mixture was used for the direct pelletization step.

### Example 7g

The process of example 7 g was scaled up in order to test the applicability of the process of the present invention in a larger industrial scale. 1,500.0 g Esomeprazole Magnesium Dihydrate, 1,200.0 g microcrystalline cellulose and 1,200.0 g Polyethylene Glycol (PEG) 4000 were wet granulated in a similar way as described in example 7b, using larger equipment. 3,640 g of the wet mass were mixed with the MCC portion excluded from the wet granulation step (1,400 g) and 224.0 g talc, 84.0 g colloidal silicon dioxide and 196.0 g Sodium Alginate and the mixture was used for the direct pelletization step. Drying was performed in a fluid bed apparatus, up to the point that the moisture of the pellets was reduced at less than 2%.

### Example 7h

The process of example 7 g was further scaled up in order to challenge the applicability of the process of the present invention in even larger industrial case. 6,000.0 g Esomeprazole Magnesium Dihydrate, 4,800.0 g microcrystalline cellulose and 4,800.0 g Polyethylene Glycol (PEG) 4000 were wet granulated in a similar way as described in example 7g, using larger equipment. 13,000 g of the wet mass were mixed with the MCC portion excluded from the wet granulation step (5,000 g) and 800.0 g talc, 300.0 g colloidal silicon dioxide and 700.0 g Sodium Alginate and the mixture was used for the direct pelletization step. Drying was performed in a fluid bed apparatus, up to the point that the moisture of the pellets was reduced at less than 2%.

Examples 7 g and 7h showed that the process of the present invention behaves in a similar way in different production scales. The output of the process concerning the quality characteristics of the pellets (pellet size distribution, shape) were similar in all cases.

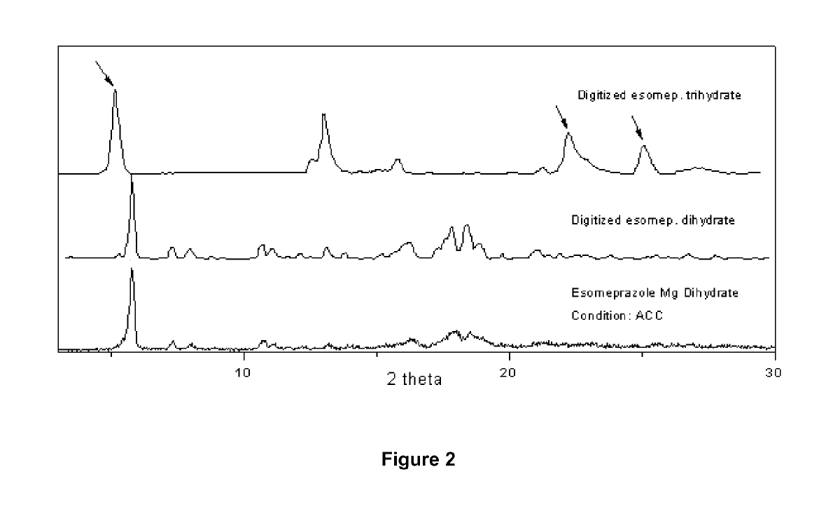

All the formulations described in examples 7a-7h released the API quantitatively in 15 minutes. In addition, a polymorph study was performed and the crystal phase of Esomeprazole Mg powder deriving from the spheroids of example 7a was established through the application of the X-Ray Powder Diffraction (XRPD) technique. The recorded XRD patterns shown in FIG. 2 were compared against the respective patterns of the dihydrate and trihydrate forms provided by the manufacturer.

From FIG. 2 it is apparent that both recorded patterns exhibit the reflections of Esomeprazole Mg dihydrate. None of the characteristic trihydrate reflections, marked with arrows in FIG. 2, could be observed.

In conclusion, with the process of the present invention rapidly disintegrating spheroids may be prepared, without deteriorating the crystal habit of Esomeprazole Magnesium Dihydrate, although aqueous media were utilized.

### Example 7i

In order to provide a comparison, conventional MCC:lactose pellets were produced via the wet granulation-direct pelletization process. These pellets released only 45% of the API during the first 15 minutes of the dissolution test.

### Example 8

Example 8 refers to the outcomes of testing the formulation of the present invention with other model drugs (Model Drug C, Model Drug D).

Model drug C is Nimesulide, an API with an aqueous solubility of approximately 10 μg/ml at 25° C. Nimesulide has been used as a model drug in numerous publications, as a result it is an excellent candidate in order to challenge the process of the present invention.

Two trials were performed incorporating different levels of Nimesulide:

### Example 8a

100.0 g Nimesulide, 500.0 g lactose, 500.0 g microcrystalline cellulose, were rotogranulated at a spray rate of approximately 30.0 g/min, at 30° C. Drying was performed at 55° C. This is the preparation process for spheroids as presented in the prior art.

### Example 8b

250.0 g Nimesulide, 400.0 g lactose, 250.0 g microcrystalline cellulose, 250.0 g Polyethylene Glycol (PEG) 4000, 50.0 g Sodium Alginate and 50.0 g Talc were rotogranulated using a similar process as in example 8a.

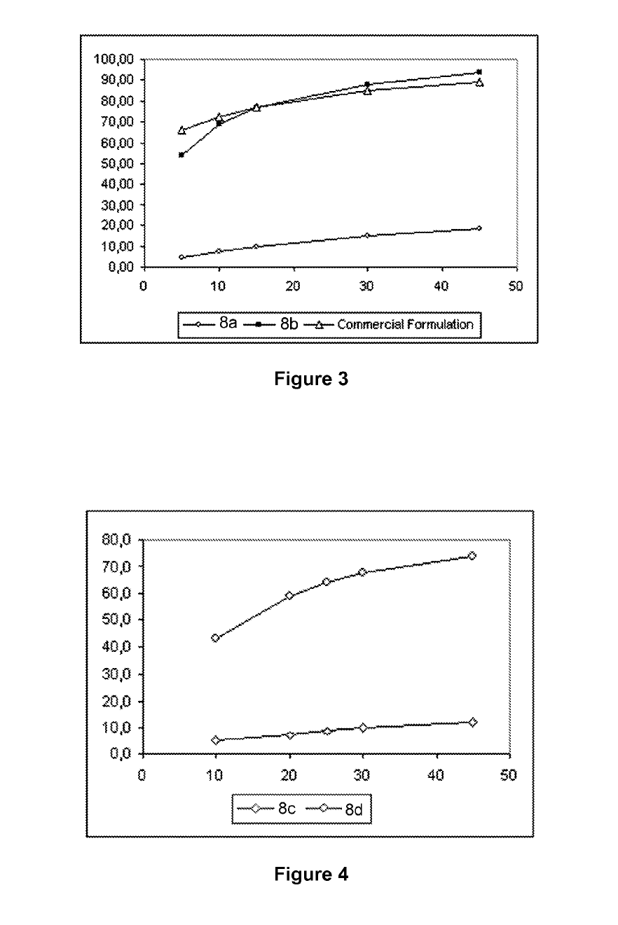

The dissolution profiles of the two formulations are presented in FIG. 3.

It is clear that the formulation of the present invention may provide an immediate release dosage form of Nimesulide. MCC:Lactose formulations as presented in the prior art fail to release more than 20% of the drug substance in 45 minutes. In addition the dissolution rate from MCC:Lactose pellets as presented in the prior art is very slow.

Model Drug D is Glimepiride, an API practically insoluble in water, typically used at doses from 1 to 4mg. Some formulation work has been recently done towards the improvement of the dissolution profile and consequently its performance in vivo, by formulating glimepiride-cyclodextrin-polymer systems (Ammar et al, 2006, Formulation and biological evaluation of glimepiride-cyclodextrin-polymer systems, International Journal of Pharmaceutics, 309, 129-138). Given that the direct pelletization process also facilitated the dispersion of low strength APIs, the approach described in the present invention is promising for the preparation of an advantageous glimepiride formulation.

Two formulations were prepared, in order to compare the release of glimepiride from typical MCC:Lactose formulations (which do not disintegrate) as presented in the prior art, with formulations prepared using the process of the present invention.

### Example 8c

12.5 g Glimepiride, 487.5 g lactose and 500.0 g microcrystalline cellulose were rotogranulated at a spray rate of approximately 30.0 g/min, at 30° C. Drying was performed at 55° C. This is the preparation process for spheroids as presented in the prior art.

### Example 8d

12.5 g Glimepiride, 387.5 g lactose, 250.0 g microcrystalline cellulose, 250.0 g Polyethylene Glycol (PEG) 4000, 50.0 g Sodium Alginate and 50.0 g Talc were rotogranulated at a spray rate of approximately 27.5 g/min, at 30° C. Drying was performed at 55° C.

A comparative dissolution profile of the two formulations is presented in FIG. 4.

The formulation of the present invention disintegrated rapidly, providing a seven-fold increase of the amount of glimepiride released in comparison with MCC:Lactose pellets as presented in the prior art.

1. A process for producing rapidly disintegrating spheroids, wherein said spheroids comprise at least one active pharmaceutical ingredient, a spheronizing aid material and at least one solid polyethylene glycol with a mean molecular weight greater than or equal to 1,000 wherein said process comprises a direct pelletization stage performed in a fluidized bed rotogranulator, characterized in that said process is carried out using aqueous binding media and in that the temperature of the process during said direct pelletization stage does not exceed the melting point of the polyethylene glycol.

2. A process for producing rapidly disintegrating spheroids according to claim 1, wherein the active pharmaceutical ingredient has a solubility selected from the group consisting of sparingly soluble, slightly soluble, very slightly soluble, practically insoluble and insoluble according to the US Pharmacopoeia.

3. A process for producing rapidly disintegrating spheroids according to claim 2, wherein the amount of the active pharmaceutical ingredient varies between 0.5 and 80%, by weight per spheroid.

4. A process for producing rapidly disintegrating spheroids according to claim 2, wherein the active pharmaceutical ingredient is a proton pump inhibitor.

5. A process for producing rapidly disintegrating spheroids according to claim 4, wherein the active pharmaceutical ingredient is selected from the group consisting of omeprazole, esomeprazole and pharmaceutically acceptable salts thereof.

6. A process for producing rapidly disintegrating spheroids according to claim 2, wherein the active pharmaceutical ingredient is nimesulide or glimepiride.

7. A process for producing rapidly disintegrating spheroids according to claim 1, wherein said solid polyethylene glycol has a mean molecular weight of between 1,000 and 10,000.

8. A process for producing rapidly disintegrating spheroids according to claim 7, wherein the amount of said solid polyethylene glycol varies between 5 and 60% by weight per spheroid.

9. A process for producing rapidly disintegrating spheroids according to claim 1, wherein the spheronizing aid material is selected from the group consisting of Microcrystalline Cellulose, Modified Celluloses and combinations thereof.

10. A process for producing rapidly disintegrating spheroids according to claim 9, wherein the amount of the spheronizing aid material varies between 5 and 80% by weight per spheroid.

11. A process for producing rapidly disintegrating spheroids according to claim 1, wherein the temperature during the direct pelletization stage does not exceed 40° C.

12. A process for producing rapidly disintegrating spheroids according to claim 1, wherein said spheroids further comprise a detackifier.

13. A process for producing rapidly disintegrating spheroids according to claim 1, wherein said spheroids further comprise at least one additional excipient selected from the group consisting of:
fillers/diluents
lubricants, glidants,
binders,
disintegrants and combinations thereof.

14. A process for producing rapidly disintegrating spheroids according to claim 1, wherein the process further comprises a wet granulation stage prior to the direct pelletization stage, said wet granulation stage including some or all the ingredients of the spheroids.

15. A process for producing rapidly disintegrating spheroids according to claim 1, wherein all the ingredients are placed in the product chamber of the fluidized bed rotogranulator at the beginning of the direct pelletization step.

16. A process for producing rapidly disintegrating spheroids according to claim 1, wherein only part of the ingredients are placed in the product chamber of the fluidized bed rotogranulator at the beginning of the direct pelletization stage, the rest being introduced in the product chamber via the powder feeder during the direct pelletization stage.

17. A process for producing rapidly disintegrating spheroids according to claim 1, wherein only part of the ingredients are placed in the product chamber of the fluidized bed rotogranulator at the beginning of the direct pelletization stage, the rest being dispersed or dissolved in the aqueous binding material, which is subsequently sprayed in the product chamber during the direct pelletization stage.

18. A process for producing rapidly disintegrating spheroids according to claim 1, characterized in that the process further comprises a coating stage, following the direct pelletization stage.

19. A pharmaceutical dosage form selected from the group consisting of capsules and tablets, wherein said dosage form comprises rapidly disintegrating spheroids produced via a process according to claim 1.

20. A process for producing rapidly disintegrating spheroids according to claim 3, wherein the amount of the active pharmaceutical ingredient varies between 0.5 and 50% by weight per spheroid.

21. A process for producing rapidly disintegrating spheroids according to claim 20, wherein the amount of the active pharmaceutical ingredient varies between 0.5 and 30% by weight per spheroid.

22. A process for producing rapidly disintegrating spheroids according to claim 7, wherein said solid polyethylene glycol has a mean molecular weight of 4,000.

23. A process for producing rapidly disintegrating spheroids according to claim 8, wherein the amount of said solid polyethylene glycol varies between 10 and 50% by weight per spheroid.

24. A process for producing rapidly disintegrating spheroids according to claim 23, wherein the amount of said solid polyethylene glycol varies between 15 and 30% by weight per spheroid.

25. A process for producing rapidly disintegrating spheroids according to claim 10, wherein the amount of the spheronizing aid material varies between 10 and 60% by weight per spheroid.

26. A process for producing rapidly disintegrating spheroids according to claim 25, wherein the amount of the spheronizing aid material varies between 15 and 55% by weight per spheroid.

27. A process for producing rapidly disintegrating spheroids according to claim 12, wherein said detackifier comprises talc.

28. A process for producing rapidly disintegrating spheroids according to claim 13, wherein said filler/diluent is selected from the group consisting of sugars, sugar alcohols, starches, resins, dibasic calcium phosphate and combinations thereof.

29. A process for producing rapidly disintegrating spheroids according to claim 28, wherein said filler/diluent is lactose.

30. A process for producing rapidly disintegrating spheroids according to claim 13, wherein said binder is selected from the group consisting of gelatins, L HPC, starches, hydropxypropylmethyl cellulose, polyvinylpyrrolidone and combinations thereof.

31. A process for producing rapidly disintegrating spheroids according to claim 13, wherein said disintegrant is selected from the group consisting of starches, celluloses, alginates, dextrans, cross-linked polyvinylpyrrolidone and polyethylene glycol sorbitan fatty acid esters, sodium carboxymethyl cellulose, modified corn starch, sodium carboxymethyl starch and combinations thereof.
